(19) United States
(12) Patent Application Publication
(10) Pub. No.: US 2025/0029194 A1
(43) Pub. Date: Jan. 23, 2025

(54) Anonemis Research Software Hard Drive Protocol Policy Processes 2220

(71) Applicant: Renee Simenona Martinez, Beverly Hills, CA (US)

(72) Inventor: Renee Simenona Martinez, Beverly Hills, CA (US)

(21) Appl. No.: 18/222,622

(22) Filed: Jul. 17, 2023

(51) Int. Cl. G06Q 50/26; G16H 20/70; G16H 50/80; H04L 9/40

(52) U.S. Cl. CPC G06Q 50/265; G16H 20/70; G16H 50/80; H04L 63/0209; H04L 63/302

(57) ABSTRACT

The embodiment of compromising software with a critical interface includes an algorithm that records micropatterns of isolated human behavior, content, and essential elements of tactical process procedures within various sections of the present application that require cyber-microexpression for its computation. Quantum Sequence Triplication is the use to achieve Quantum Supremacy without grey area bias.

### FIELD

The present application relates to the embodiment compromising of a software application process in quantum behavioral science regarding specialized observation monitoring and methodology procedure with critical outcome processes, as it relates to a specialized encrypted algorithmic database and quantum process procedure. The stated relates to a software application and isolated behavioral methodology procedures, as it relates to cognitive science and various tactical areas. This software application includes a user-interface with a customized operating system.

This software application encompasses a customized operating system which yields to areas of telecommunications, automotives, transliteration development, chip technology, communications, data analysis, data algorithm structure, mechanical engineering, behavioral science, medicine, behavioral science, nuclear science, space technology, defense, quantum approximation and any software devices offering access to information.

### BACKGROUND

Cyber-Micro-Expressions with Quantum Sequence Triplication and Quantum Operation Supremacy is an embodiment comprising of a specialized algorithmic software process. The stated software process is a multioperation system which runs on a quantum software process database and a methodology which data source references are critically verified information by Quantum Sequence Triplication.

Quantum Sequence Triplication where the use of three qubit bits to achieve Quantum Supremacy without grey area bias. Qubit 1 and 2, are encoded using the Quantum Operation Supremacy methodology, except for the data being opposing information data. Finally, qubit 3, is the output of the data calculation process of opposing outcomes to determine the sum data devoid of grey area but most importantly, fact checked. This system database uses current and real-time information which is vetted within the system queue for verification by both automated algorithms and operator oversight.

Cyber-microexpression is an embodiment of an algorithmic software and an isolated behavioral observation pattern recognition system in which the subject or contents micro-pattern behaviorism are monitored in the scope of involuntary reactions between the human subconscious and machine, and that action is displayed by cyber-microexpressions.

In addition, when humans make facial expressions, we create the emotions attached to that expression to determine a nonverbal response. These elements of psychology, as expected, are interpretable not only in the digital spaces but in various tactical fields.

Further objects, features, advantages and properties of this software and methodology according to the present application will become apparent from the detailed description.

### SUMMARY

This embodiment of the compromising software with a critical interface includes an algorithm which records micropatterns of isolated human behavior, content, and essential elements of tactical process procedures within various sections stated within present application, requires of cyber-microexpression to achieve such computation.

The disclosed exemplary embodiments provide a software and process behavioral procedures for such tactical purposes.

In one embodiment the software's operating system is developed with an interactive interface which analyzes micropattems in a specialized database which consists of academic research and other sources to remain current. This database allows the algorithm to optimize its operation in a specialized sequence which differs from the traditional or current popularized databases.

The use 3 qubit bits to achieve quantum supremacy without grey area bias. This feature is essential for monitoring intrusions or malfunctions in a multitude of systems in various sectors. The rapid shuffling process is a specialized technique which allows swift detection in any mechanism, process procedure, or operation.

### BRIEF DESCRIPTION OF THE DRAWINGS

In the following detailed portion of the present description, the teachings of the present application will be explained in more detail with reference to the example embodiments shown in the drawings, in which:

No FIGURES Included.

### DETAILED DESCRIPTION

In the following detailed description, the software process according to the teachings for this application in the form of technical science will be described by the embodiments. It should be noted that the teachings of this application can also be used in any other sectors such as: telecommunications, automotives, transliteration development, solar, chip technology, communications, data analysis, behavioral science, transportation, medicine, nuclear science, space technology, defense, quantum approximation, data algorithm structure, mechanical engineering and any software devices offering access to information.

The first embodiment comprising of stated system that works by implementing a specialized rapid algorithm process which correlates data in real-time and produces predictive data which is precisely relevant to the case. The stated software process operates on a single quantum system, therefore allowing the optimal sequencing and tactical outcome.

### Observational Embodiments

For example, subject A, is an actor who is experiencing a psychotic break on Twitter and subject B is tweeting inappropriate content to provoke further response with subject A. Those directly and indirectly involved with subject A, may display involuntary reactions by digital footprints displayed in their digital microexpressions. This is cyber-microexpressions.

### Cyber-microexpressions Methodology:

Isolating the individuals involved on a separate observation account for observational evaluation.
Monitoring by video and persons of the behavioral interactions to establish a normal baseline in behaviour vs. when engaging in criminal activity which causes a change within the behavioral response which is acted out subconsciously.
Establishing these behaviors in a quantitative measurement as it relates to psychological criminology, cognitive reactivity and incorporating the methodology of Rorschach prompts.
Reactivity and Patterns: The calculating of the reactivity stimuli with each subject when engaging on social platforms is critical in Cyberstalking, Terrorism, Cyberbullying cases. Establishing behavioral patterns during subconscious engagements to further determine behaviour patterns used when engaging in criminality but most important, log those as video evidence.
The Prompt: Inducing behavioral responses with the use of prompts, Input. Once the subject has been evaluated with the reactivity and patterns profile, law enforcement or military personnel can then send content specific to regarding those trigger prompts to induce a reactive response (output) for evaluation.

These involuntary responses are critical in providing information regarding crimes which are committed in “silence,” which makes this software a fundamental element in cybersecurity as well as terrorism.
The Report: Upon completion of these tests the administrator will be able to establish the next pattern of behaviour the subject will make without having to utilize a prompt.

The use of the Rorschach prompt methodology has changed the way the user interacts with the operating system, thus, conditioning their cognitive understanding and behavioral responses to react as well as engage with content or users regarding suspected crimes.

Thus, allowing the administrator to be many steps ahead of the subject(s) cognitively, and most effectively, monitor the subject now subconsciously acting out these cyber-micro expressive behaviours of criminality, already established within the subjects patterned profile. Thus, producing criminal evidence or a silent confession which is video reported.

This is critical in preventing crime as well as providing in-depth research in the field of criminology. Observing and applying this methodology in group a group setting is effective regarding behavioral monitoring and successful output data.
Research data obtained from cases will be inputted in the Database to quantify these patterns, continuous adjustments are critical in building stronger algorithms within the software which can detect these behavioural pattens without the guidance of an administrator. This software can run concurrently with any operating system, telecommunication devices, or social platform.

The software will then isolate these accounts to monitor by video recording. This process is intended for analysis of data output regarding future prompts and evaluation, research, and adjustments.

Cyber-microexpressions can manifest based on the emotion attached to expression or overall feeling or mood of a subject. These outputs can be independent or reactive. In this instance, we are focused on the reactive type. If the collective reaction towards Subject A's psychotic's break is contempt, the trigger event will elicit an emotional response in those subjects which will result in cyber-micro expressive responsivity in subsequent posting and other online actions, reflecting such emotions, regardless of algorithmic content because the subjects intentionally post consciously sought content.

This is also particularly prevalent in cases of online feuds as well as cyber-bullying; the perpetrator will post content to elicit a negative emotional response from the victim. The perpetrator posts to tell a story on their cyber channel and gain not only a response but also an induced narrative to influence “public opinion” in their favor. For example, if Subject X and Subject Y are feuding behind closed doors, they may act out passive aggressively online, known colloquially as “throwing shade” or “shading.” This is a way to subtly act out aggressions, whether other onlookers are aware of the feud. Thus, Subject X may, for example, post something on social media to gain sympathy and/or villainize Subject Y.

It is not uncommon for Subject X and Y to engage in competitive behavior online: Subject X posts a selfie, so subject Y posts one as well, with the aim of regaining the public gaze, as if to say, “look at me, I am beautiful, too.” These are age-old human reactions, but they are now being acted out online.

In the case of high-profile figures, in this instance of Subject A, the other individuals are brands, self-aware of public observation of their online behaviour, thus, they avoid liking content that can be linked to incriminating behaviour, but their activity will be live during the psychotic episode of Subject A.

In other forms of harassment, the Subject will follow target on all platforms, even if the target no longer is engaging. Both sides are classically conditioned to every dimension of conflict. The aggressor, as we see with many narcissistic personality disorders, works very diligently at provoking the target to act out so, the subject can feel gratification and control over the target, whom they now view was weak. The subject will often use a side account often referred to as “Alt accounts.”

This allows the subject to move anonymously. However, both parties experience an increase of onset anxiety and depression and other negative reactions, such as eating disorders as a result. The attacker will increase internet use whether it involves obsessing, re-reading, or strategizing. This can result in less time updating their own personal page because they become consumed in the alternate reality which they must maintain to continue provoking the target. In addition, the aggressor may begin to consciously or subconsciously mlrronng the target, by copying their physical appearance, personality traits and/or type of online content.

Again, because these actions are cyber-microexpressions, they may be difficult to decipher, and may require more in-depth observation.

This type of competition can become increasingly hostile in instances of romantic competition. Let us assume that Subjects X and Y are female love interests of Subject Z. Subject X is an ex-partner, while Subject Y is a new, current partner of SubjectZ. Subject Xmay attempt to gamer Subject Z's attention due to jealousy over the new relationship by posting provocative or attention-seeking content to gain interest and or sympathy from Subject Z. Subject Y may then choose to respond in competition, as well. This dynamic can be further complicated, just as offline “love triangles,” if Subject Z has interest in both parties. Unhealthy triangulation can then play out online as well as offline. As this example displays, some online interactions which may not be illegal can still have adverse effects on both the victim and the perpetrator, especially such instances of unhealthy competition.

The use of algorithms can be a dangerous ally to mental illness in the case of subjects experiencing psychosis, delusions, or other disconnections from reality. The A.I. predictive content can induce paranoia, aggravating delusions related to bipolar or schizophrenic episodes in mentally ill individuals because their social media feed may be flooded with content related to their delusions.

The linkage of the study of cyber-microexpressions with gathering evidence of crime online can help close the loop of the internet injustices that are currently slipping through the cracks.

The digital footprints of perpetrators will become key in the future of tracking and apprehending online criminals. We can go beyond IP addresses to understand the in-depth movements of harassers and stalkers. Even malicious hackers are currently welcomed with far too many gaps in security, which need to be tightened up.

### The Algorithm Embodiment

A qubit or quantum bit is a basic storage/symbol, in which a bit of quantum information is stored/encoded-the quantum version of the classic binary bit physically realized with a two-state device. In Quantum Sequence Triplication, we use 3 qubit bits to achieve quantum supremacy without grey area bias.

Qubit 1 and 2, are encoded using the Quantum Operation Supremacy methodology, except for the data being opposing information and data. Finally, qubit 3, is the output of the data calculation process of opposing outcomes to determine the sum data devoid of grey area but most importantly, fact checked.

The technology is intended for military strategy and defense, medicine and medical development without error, and the groundwork for quantum supremacy.

Quantum Operation Supremacy. The use of traditional binary single qubit output methodology is the baseline for this operation and in the alteration regarding quantum operation. The system works by implementing artificial qubits to source and produce calculation data for equation execution.

The data is stored and categorized into more ridged data columns to eliminate error and to achieve simplification. Sorting data by patterned repetitions in the level of importance relevance for desired outcome. This rapid data shuffling and detecting repetition sources regarding patterns can assist solving complex cryptology. Artificial qubits have infinite potential for quantum calculations. The use of frequency detection can detect slight use, alteration, or manipulation within the quantum monitoring system. This is an advancement towards code breaking within algorithmic systems such a reversal of Shor's algorithm, as well as CAPTCHA generated processes.

This embodiment comprises of the stated software and methodology according to claim 1, Quantum Cyber-Micro-Expressions Computation Research and Adjustments: Diagnostics: Medical Healthcare Treatment, Criminality, Education, Law Enforcement and Military

Quantum Cyber-Micro-Expressions Computation is the use of Quantum computing methodology analysis of the observational behavior data of non-verbal subconscious involuntary reactivity frequency. Quantum Cyber-Micro-Expressions computation calculates the reoccurrence or repetition in behavioral pattern to determine analysis output diagnostic.

Calculating the assessment evaluation data within The Rorschach Cyber-Micro-Expressions Algorithm Database with the use the Quantum computing methodology of the observational behavior data analysis of non-verbal subconscious involuntary reactivity frequency.

Quantum Cyber-Micro-Expressions Computation, which calculates the reoccurrence or repetition in behavioral patterns. The data is then computed and analyzed during The Rorschach Cyber-Micro-Expressions Data Analysis, which prompts the procedure of comparative analysis processes, using the database as a baseline reference for diagnostic detection computation prototype.

The data sequences obtained from the database can be quantified then compared to the new findings regarding the subject's data using the same metric as a baseline, thus producing diagnostic results.

It is then grouped together into one report with the results sent to the psychiatric examiner for final analysis of findings, then finally upon completion, a detailed report is sent to the respect parties concerning the patient findings and analysis

A software and methodology according to claim 1, compromising of the use behavioral isolation observation and specialized algorithm to determine computation results. The Rorschach Quantum Cyber-Micro-Expressions Algorithm Computation software is an intended to be used for educational studies regarding the United States government, law enforcement, healthcare industry, education, military, quantum computing research. The software requires highly meticulously adjective information input.

The Rorschach Quantum Cyber-Micro-Expressions Data Analysis

The Rorschach Quantum Cyber-Micro-Expressions Data Analysis collected is processed in a comparative behavioral analysis database.

The processing of psychoanalytical data to compute calculations and determine evaluation results on subjects' reactivity responses to related content and data in association to computed content formulated by The Rorschach Quantum Cyber-Micro-Expressions Algorithm.

The Rorschach Quantum Cyber-Micro-Expressions Algorithm Database is an index of psychiatric behavioral response data provided by expert medical advisory, research, data regarding past cases and consented patient records, controlled studies, and scholarly data in association. The Rorschach Quantum Cyber-Micro-Expression Algorithm Evaluation. The Rorschach Quantum Cyber-Micro-Expressions Algorithm Evaluation is the process of translating the findings of The Rorschach Quantum Cyber-Micro-Expressions Machine Learning

Algorithm to evaluate the subjects' reactivity response to related content or data in association with case and evidence.

This requires analyzing records of behavioral data in the database; previous acquired data, studies, and research to compare to the current findings of the patient for a comparative analysis of The Rorschach Quantum Cyber-Micro-Expression Algorithm results, to formulate a final evaluation assessment.

### Quantum Cyber-Micro-Expressions Computation

Quantum Cyber-Micro-Expressions Computation is the use of Quantum computing methodology analysis of the observational behavior data of non-verbal subconscious involuntary reactivity frequency.

Quantum Cyber-Micro-Expressions computation calculates the reoccurrence or repetition in behavioral pattern to determine analysis output diagnostic.

Calculating the assessment evaluation data within The Rorschach Quantum Cyber-MicroExpressions Algorithm Database with the use the Quantum computing methodology of the observational behavior data analysis of non-verbal subconscious involuntary reactivity frequency. Quantum Cyber-Micro-Expressions Computation, which calculates the reoccurrence or repetition in behavioral patterns. The data is then computed and analyzed during The Rorschach Quantum Cyber-Micro-Expressions Data Analysis, which prompts the procedure of comparative analysis processes, using the database as a baseline reference for diagnostic detection computation prototype. The data sequences obtained from the database can be quantified then compared to the new findings regarding the subject's data using the same metric as a baseline, thus producing diagnostic results. It is then grouped together into one report with the results sent to the psychiatric examiner for final analysis of findings, then finally upon completion, a detailed report is sent to the respected parties concerning the patient findings and analysis. The Case of Cyberstalking. In the case of criminality, The Rorschach Quantum Cyber-Micro-Expressions Algorithm Computation would be paired with third-party social media platforms such as Instagram and Twitter. The Rorschach algorithm is designed to record the subjects' behavioral patterns.

The Rorschach Quantum Cyber-Micro-Expressions Algorithm Computation m all other occurrences is offered as a software requiring installment and continuous configurations. In the case of cyberstalking, cyber-crimes, criminal and terrorism monitoring, The Rorschach Quantum Cyber-Micro-Expressions Algorithm Computation would quarantine all parties involved in an observation mode, which would isolate the activity of those parties who are alleged to be involved in the crime, including the victim. This process digitally records actions and reactivity behaviors on all subjects.

The process of calculating the assessment data within The Rorschach Quantum Cyber-Micro-Expressions Algorithm Database with the use the Quantum computing methodology of the observational behavior analysis of non-verbal subconscious involuntary reactivity frequency. The Quantum Cyber-Micro-Expressions Computation, which calculates the reoccurrence or repetition in behavioral patterns. The data is then computed and analyzed during The Rorschach Quantum Cyber-Micro-Expressions Data Analysis, which prompts the procedure of comparative analysis processes, using the database as a baseline reference for diagnostic detection computation prototype.

The data sequences obtained from the database can be quantified then compared to the new findings regarding the subject's data using the same metric as a baseline, thus producing diagnostic results. It is then grouped together into one report with the results sent to the psychiatric examiner for final analysis of findings, then finally upon completion, a detailed report is sent to the respected parties concerning the patient findings and analysis.

The Rorschach Quantum Cyber-Micro-Expressions Algorithm Computation by Sector Military Procedure and Protocols Action Analysis Data regarding previous deployments, captures, terrorism profiling, enemy protocol, strategy operational offense and defense behavioral analysis.

Education, Mental Health, and Social Analysis Analyzation of student's workflow stylization and learning preference; visual and auditory.

Monthly evaluation to determine students' emotional well-being, which is overseen by school psychologist.

This methodology introduced in a safe zone processes and procedures will encourage students to be honest and with the use of A.I. computing testing of the subconscious will assist in evaluation.

This data is crucial in the prevention youth suicide as well as school shooting and mitigating bullying.

It is required that the school board protect the privacy of the students and participation requires parents' consent to engage.

### Healthcare Analysis

Potential diagnostic of mental illnesses based on comparative data and research. The Rorschach. The Rorschach, an ink blot test is a personality test that involves the evaluation of a subject's response to ambiguous ink blots. This test was published in 1921 by Swiss psychiatrist Hermann Rorschach. The interpretation of people's responses to the Rorschach Inkblot Test was originally based on psychoanalytical theory but investigators have used it in an empirical fashion. When this test is used empirically, the quality of the responses is related to the measurements of personality to become more agreeable to ideas and advertising. When applied in neuroscience: psychiatry, cognitive operation of adopting ideas and understanding perspectives, thought processes and the alteration of one's baseline of comprehension within equilibrium and therefore, altering existence or actuality in induced state. The use of this methodology assists in digital psychological warfare propaganda which is prevalent and furthers the educational uses regarding neurological science.

The topics include the science of trolling, passive aggressive expressions, nonverbal communication, celebrity worship, and cyber harassment among other topics. This report delves into the patterns of involuntary subconscious responses to individual's actions and the awareness of social circumstances as they relate to offline behavior.

The study examines the marriage between conscious outward social media actions, such as public posts, and subconscious actions, including likes and secondary behaviors, and the overall relationship of these digital footprints which can be linked back to cyber harassment and other crimes”

This embodiment comprises of the stated software and methodology according to claim 1, compromising the use behavioral isolation observation and specialized algorithm to determine computation results. A.I Cluster Chain Diagnostics in Social Platform Psychiatric Disorders, Terrorism and Criminality.

The use of smart A.I machine can create sequencing behavioral data to identify behavioral clusters within the framework of the classification regarding psychiatry, terrorism, and criminality.

The software categorizes clusters in a behavioral sequencing and those identical sequencing classifications are applied in the context of terrorism. Establishing which personalities have a higher predisposition of radicalization and pinpointing the source association within their criminal enterprises.

The programmed behavioral mechanism in methodology is monitoring behaviors associated with mass homicides, school shootings, patterns in aggressive behaviors with race and culture accounted within the construction of the programming to eliminate misdiagnosis and irrelevant leads.

Furthermore, reverse engineering is engaged by inserting data-collection malware in high-risk users to accumulate and categorize overall digital footprints to provide a clear overview on how to classify on-going procedural within Homeland Security infrastructure.

This embodiment comprises stated software and methodology according to claim 1, comprising the use behavioral isolation observation and specialized algorithm to determine computation results.

This embodiment comprises stated software and methodology according to claim 1, Therapy Behavioral Conditioning with VR Technology to enhance the use of algorithms.

Utilizing algorithms, as it stands, can create addiction, which means it has the potential to also reverse it. It can also help with ptsd, memory, retraining the brain, repair some aspects and/or extend capacity by monitoring behavioral patterns then altering it through conditioning.

Algorithms can be useful in treating depression by simply observing the user's behaviors to eventually suggest positive images, outcomes, and feedback. Even creating personalized simulations to help elevate moods, work through triggers, and change perspective on atmosphere. All accompanied by medication and therapy under physicians' care.

Conditioning regarding cyber threats by introducing series of algorithmic patterns then observing the user's voluntary/subconscious patterns to determine intent, affiliation, desires, vices, religion, and association. This would be done by turning the algorithms on and off in intervals while observing the subject's behaviors. This would be used in conjunction with my innovation of cyber-micro expressions. It would also help us better understand how the brain works.

In addition, changing the way the human brain responds to algorithms will have a positive outcome on how users respond to advertising algorithms. It is about changing consumers' perspectives of advertisements, not the consumers themselves.

This embodiment comprises stated software and methodology according to claim 1, AR De-radicalized Conspiracies Theories. Utilizing the same algorithmic format of locating ‘like-minded’ users who believe and support the same dangerous conspiracy theories and replacing inaccurate information with facts in the matter in which they learned, they will unlearn.

This will monitor affected users purchases of firearms and search history to prevent violence and/or civil war. It will compile the most affected and at-risk users for violence for law enforcement observations. For persons under 16, the (u16 users), parents must review branding agreements, with signature. The significance of this is accountability and liability protection. This tool will verify underage users preventing unauthorized access to u16 users by adults. Parents can review ads their child has watched and choose to opt out of certain types of advertising.

This embodiment comprises stated software and methodology according to claim 1, AR Youth De-radicalized Conspiracies Theories. Utilizing the same algorithmic format of locating ‘like-minded’ users who believe and support the same dangerous conspiracy theories and replacing inaccurate information with facts in the matter in which they learned, they will unlearn.

This will monitor affected users purchases of firearms and search history to prevent violence and/or civil war. It will compile the most affected and at-risk users for violence for law enforcement observations. For persons under 16, the (u16 users), parents must review branding agreements, with signature. The significance of this is accountability and liability protection. This tool will verify underage users preventing unauthorized access to u16 users by adults. Parents can review ads their child has watched and choose to opt out of certain types of advertising.

Utilizing the same algorithmic format, when detecting depression amongst u16 users who are prone to being bullied or cyber-bullied, a report is sent to parents. This tool is essential for preventing school shootings and suicides. It is then the parents' responsibility to notify the school administration office. Early prevention is essential when a child has become a danger to themselves or others.

For users, logging off each session will be prompt with an Emotional Impact Tool: Cool Down. Users will be prompted every time during sign off. This prompt is formed a calculated on a user-by-user basis, according to their digital footprints in said app. The user will be shown a set of images to induce a calming effect, positive affirmation, and feedback. A long-debated suggestion is removing likes. This will be implemented now. We will also create built-in features to notify when a picture has been edited to a certain degree. This removes the competitive aspects and anxiety surrounding that feature, which causes such addiction.

Users are unsearchable within social media platforms. They can send requests but cannot receive them from people over 18 without a verification process.

This is an equally important and exciting initiative where social media challenges will start incorporating the use of outdoors. The act of incorporating outdoor time into social media use is essential for the mental health of all users of every age. The simplest and most effective way to ensure participation is to use brands, trends, and celebrities to push the process.

Challenges can incorporate filming 5 minutes of bird sounds or meditating in nature for 5 minutes. Increasing popularity of remote hiking groups and guided meditation. Another example of this is jumping rope or online dance parties to promote physical fitness for users while offering another opportunity for product placement.

Social Media Advertisers Reconstruction Scale. ADVERTISING Model: Parents can review ads their child has watched and choose to opt out of certain types of advertising. Parents reviewing branding agreements allows advertisers to connect on a deeper level with consumers who now have their own accounts connected to the applications.

This embodiment comprises of the stated software and methodology according to claim 1, Computerized cognitive therapy to treat Internet psychosis, change patterns of thinking, and other cognitive disorders and/or syndromes. Utilizing a series of patterns and tests to diagnosis the characterization of the internet induced psychosis, the patient is treated with a series of patterns which induces the cognitive reversal through conditioning. It records the patients' thought and learning patterns. This method can both induce and treat psychosis and thought patterns. This therapy requires an immersive experience assisted with VR type glasses to engage the obsessive patterns which this diagnosis requires.

Cognitive restoration is also a defining feature in this treatment, increasing recall of data and information. Emotion can be induced using this method associated with false memory in the event of warfare. The therapy is developed on a personalized plan for individual patients through the collaboration of Psychologists, Psychiatrists, Neurologists, and other Physicians.

This embodiment comprises of the stated software and methodology according to claim 1, AR Quarantine Software and Hard drive is to combat against social engineering attacks which originate by user error in download or malicious links. This is an application which acts a third-party vetting to any files or web prompts which reroutes the client to a separate hard drive where information is decoded, scanned, and re-encrypted back to the main network, only if determined safe for viewing and delegated system access.

This embodiment comprises of the stated software and methodology according to claim 1, compromising the use behavioral isolation observation and specialized algorithm to determine computation results. Department of Defense, Department of Homeland Security and Federal Aviation Association. Federal Generation Dynamics Operations Communications System.

In the event of an outage or threat in the FAA or systems processing and monitoring tools operating in similar processes, the Federal Generation Dynamics Operations Communications System can be engaged.

This moderation system is overseen by Navy intelligence in conjunction with Homeland Security and The Department of Defense, stationed on both foreign and surrounding territories unaffected by outrages or threats.

This system operates on the backup generators which houses by proxy servers that pilots, radar, satellite, inferred technology that manages the monitoring and detection systems, as well past behavioral data to ensure safe grounding of aircrafts in mid-flight and the seamless continuation of patrolling on both foreign and US territories.

This generator is connected by proxy with secure access to up to date shared, past, and current data on a separate server for security, terrorism measures, and no weather-related outrages yet to be classified by officers.

The officers located outside of the affected zones can access the system and track intelligence data from the generators, radars, satellite, inferred technology of authorized and non-authorized aviation jurisdictions traffic as seen fit. These systems are effective in the case of weather-related outages. These systems can be used on an ongoing basis to gather intelligences, particularly with the AnonemiX Media Frequency and eventually with the implementation of systems such as Solar Satellite Telecommunications Network and the Solar Transportation Network, as primary or secondary protocol measures which will be highly instrumental in these processes and procedures.

The use of the Department of Homeland Security Sociologic Behavioral Profile Tracking and Alert System of inputting prior data from news circuits, high engagement content, sociological factors such as protests, revolts, and subsequent casualties for offensive tactical operations. As well as, tracking foreign trade deal activities relating to the United States Territories. As seen in Behavioral Science of Online Harassment: Cyber-Micro-Expressions, Bullying and Stalking on Social Media.

This embodiment comprises of the stated software and methodology according to claim 1, compromising the use behavioral isolation observation and specialized algorithm to determine computation results. Department of Homeland Security. Sodologic Behavioral Profile Tracking and Alert System.

Monitors suspicious activities on social media networks such content with high views and engagements but lacking action driven output such as likes and comments. This is a result of hoovering activity, often seen in attempts to observe intelligence undetected. Data can be pulled from social media enterprises in the event of attacks to verify engagements, surrounding users and that data regarding online behavioral pattern. As seen in Behavior-al Science of Online Harassment: Cyber-Micro-Expressions, Bullying and Stalking on Social Media Inputting prior data from news circuits, high engagement content, sociological factors such as protests, revolts, and subsequent casualties. Tracking foreign trade deal activities relating to the United States Territories.

This data acts as a monitoring system to determine the likelihood of an attack upon American soil in attempts in preventing sociologically behavioral patterns leading up to such catastrophic events. Monitoring high-risk social media accounts who engage in radical issues relating to terroristic offenses as it relates to the United States Territory. Utilizing the data system accumulate data report to immediately notify Homeland Security and Department of Defense.

This embodiment comprises of the stated software and methodology according to claim 1, compromising the use behavioral isolation observation and specialized algorithm to determine computation results. Cyber-Micro-Expressions Special Forces Detection Integration Software (Terrorism and Missing Children)

The natural progression in the digital age is to require all social media platforms to share user interaction concerning missing children and terrorism content in areas of interests, as hoovering is a tactic used in cyberstalking and criminology behaviorism. This software is a third-party integration used by law enforcement and military personnel.

This embodiment comprises of the stated software and methodology according to claim 1, Safe Medical Cyber-Security Solution offers security monitoring software for patients with wireless implanted or wearable medical devices. Cybersecurity is one of the upmost important concerns in an almost purely digital world, meaning that up-to-date and secure configurations are nonnegotiable.

Safe Medical Cyber-Security Solution offers peace of mind by implementing A.I machine behavioral monitoring, intrusion detections, quarantine servers, data backups, device data operations via an offline secure secondary server data collection and ongoing anti-virus configuration. Safe Medical Cyber-Security Solution implements A.I machine monitoring, which tracks the device operations, user behaviors and alerts the user of a usual activity which is cause for concern. When the A.I systems determines the presence of usual activity, it will transition the device into incognito mode, which will initiate the intrusion detection and protection procedures, in addition to switching the device to a secure secondary backup network server which is separate from the primary server in question, which could be compromised.

This protocol is a critical offensive response to a threat because it prohibits an attacker from the gaining further access. In the event of an intrusion, the medical device can use the backup server which IS provided by Safe Medical Cyber-Security Solution. During this process, the medical manufacturer will be notified and data collection from the patient's device will not be affected but collected via the secondary server. When the event is resolved that data input is transmitted back to the device for data consolidation. Upon completion, a primary report is then sent to the manufacturer, physicians, the cybersecurity division in law enforcement and an overview is sent to the patient which will revisit patient protocol for protecting data.

The methodology is important because it allows rapid and consistent monitoring of devices in real time in the case of malicious intrusions. This alert can be lifesaving.

This embodiment comprises of the stated software and methodology according to claim 1, compromising the use behavioral isolation observation and specialized algorithm to determine computation results. The Department of Defense. Quantitative Strategic Operation Defense System (Q.S.O.D)

The quantitative strategic operation defense system trains military agents on defense responses using Quantum Sequence Triplication and Quantum Operation Supremacy methodology.

The program analyzes various combat sequences from classified military footage information data, research information data, game theory ideology and Nash Equilibrium for optimum military strategical defensive as well as offensive outcomes in an interactive A.I interface with a training pod.

The system is virtual reality-based simulation in pod, equipped with weather, defense, offensive, and appositive reaction, and real-time outcome for military training.

The Department of Homeland Security: Elective Mental Health Junior Military Logistic Analyst and Veteran Research Program

Elective Mental Health Junior Military Logistics Analyst and Veteran Research Program is an innovative program in which military agencies recruit teens who have been rigorously evaluated by mental health professionals for eligibility.

The applicant must meet a certain standard of criteria, i.e., a certain number of behavioral hospitalizations and diagnostics.

Military recruiters can opt to participate in this program which requires leading school assemblies to speak to teens about school shootings and develop an opportunity for opening communication lines regarding the heavily documented interests in assault rifles involved in school shootings. Thus, providing a healthier alternative for recruits who have diagnosis with and/or behavioral problems which psychotic violent features. This program will not be operated on base, nor will military clearance be granted.

As noted with many school shootings which often end in self-inflicted outcomes, these recruits will be essential for providing predatorial natural instinct-based knowledge for future defense operation research data. If the recruit is over 18, they then can opt to participate in special forces assignments with mirrored outcomes.

In the case of long-term recruits, they can provide insight into infrastructure building tactics regarding natural born predatorial instinctual hunting strategies which their condition causes them a predisposition to.

The data obtained will be used for The Department of Defense Quantitative Strategic Operation Defense System (Q.S.O.D).

This program encourages veterans to participate in the on-going research study operation who have development c-ptsd which violent psychotic features and urges, for ongoing observation and healthcare in exchange research study.

The goal is to understand these disorders by way of research and build better relationships within the community.

This embodiment comprises of the stated software and methodology according to claim 1, compromising the use behavioral isolation observation and specialized algorithm to determine computation results. C.M.E Criminology Engagement Prompts and Identification Quarantine Methodology. This process of procedural tactical operation has data which reflects seven years of its conception with the use of scientific methodology regarding innovation.

This is a methodology and software which provides physical evidence over the course of time to assist the Department of Justice in the prosecutions of cyberstalking, hacking, and the decoding internet subbing down to a science concerning criminology.

The crucial elemental baseline for this operation is the Quantum Cyber-Micro Expression Methodology. This methodology identifies the key aggressors within cyberstalking, espionage, terrorism, hacking, and infiltration within a digital format or computer networking system concerning government operations.

This system software is assisted by A.L and M.L.

In the event of a hacking, a key and required action of this methodology is to isolate the suspected criminals on an observation account to observe their interactions, both independently, as it concerns the crime, and regarding accurate digital FBI profiling. This is a tactical response to not further compromise sensitive information on the server by using a separate space. Prompts are employed to garner a response which effectiveness has been observed in the early research phase, this phase was approximately tested for seven years using the scientific methodology. This is strengthened by the sequencing of reactive responses and their relevancy over the series of seven years.

The following prompts were employed for the following cyberstalking obsessive criminals:

### Subject/Trigger

A/Jokes
L/Emoji.
Y/Blue
Z/Dogs
W/Rocks
P/Selfies
D/Writing

These prompts were tied to events which happened in the past, in which the victim and criminals were both involved but there were unwarranted assaults inflicted upon the victim.
Universal Prompts
New Pictures/Selfie

In the on-going study using the scientific methodology, the sequenced behaviorism regarding each subject was recorded.

Thus, upon the revisiting of Group A on a significant event would indict whether the criminals were still living within the rumination under the delusion induced micro-aggressive communicative behaviorism to assault the victim.
Group A: I have made no mention of it for over a YEAR.
Still doing the same tactics to gamer attention as the other group. (Group Band Group C

Group A, the criminals were still ruminating without prompt (one sided conflict), interaction (clear indication of cyberstalking and hacking). However one-sided, they were still spiraling, cycling in the same abusive behavior, and exhibiting the same mental health issues with destructive patterns as well as features of racism. In short, attacking the victim without reason, prompts or motive. They continued to obsessively stalk and engage the victim, reinforcing reality to their delusional thinking. Often seen in stalking cases, as stalking is a sociopathic behavior in it of itself.

Most significantly, the evidence of hacking by the criminals reciting the information left in the isolated communication log, via trolling, posting, subbing, and reciting back the misinformation as a confessional, which was relayed in the chat for that very reason. The methodology of purposefully distributing misinformation to identify the vulnerabilities within the communication sourcing to identify a leak, or espionage.

This methodology identifies an aggressor, cyberstalking, espionage, terrorism, hacking, and infiltration within a digital format or computer networking system”

This system software is assisted by A.L and M.L

Below are the findings:

Group A, Group B, and Group Call acted out the same behaviorism when intentionally prompted or not. Thus, creating a domino effect in proving the vitality in each.

This is supported using Anonemis Research, LLC, innovation of tagging, spotlight content labeling methodology, and algorithm manipulation of non-verbal communication methods Innovation of tagging content to users' accounts for communication.

Spotlight content labeling by highlighting fictitious users. Algorithm manipulation non-verbal communication methods. Otherwise known as, Two-way Cybernetic Morse Codex Communication.

This embodiment comprises of the stated software and methodology according to claim 1, compromising the use behavioral isolation observation and specialized algorithm to determine computation results. Digital Identity Chip: The idea of replacing traditional passwords with encrypted data chips for secure user identification is a concept that aligns with advancements in authentication and cybersecurity. This approach aims to enhance security and address challenges posed by cybercriminal activities facilitated by anonymity. Below is a revised version of your statement:

“Substituting conventional passwords with encrypted data chips, personalized as secure identification codes for each user, is a progressive approach to safeguarding personal data. This innovative method not only bolsters security but also plays a crucial role in combating cybercriminal activities that thrive on anonymity. The use of encrypted data chips provides a robust layer of protection, significantly reducing the risk of unauthorized access and ensuring a more secure digital environment.”

This embodiment comprises of the stated software and methodology according to claim 1, compromising the use behavioral isolation observation and specialized algorithm to determine computation results. During the process of Computative Persuasion Conditioning Therapy, the subject is shown specialized content which is relevant to the desired persuasion conditioning outcome unconsciously, while operating the platform and device. This process requires the reconfiguration of the telecommunication notification system alert operation actions in the cellular device. The purpose of this procedural operation and reconfiguration is intended for the ability of transmitting specialized communications to recipients regarding the persuasion conditioning process, while adopting a similar methodology used in targeted advertising. Computative Persuasion Conditioning Therapy dispatches selected specialized content using an algorithmic format to strengthen the user's connection to the cellular environment by reconfiguring the cellular notification alerts on every day social media platforms and applications to proselytize indoctrination of desired persuasion conditioning outcome. These processes and procedural operation are accompanied by a moderator agent transmitter who is stationed at a base, operating the administrative access controls to the telecommunication layer content database, which can also operate manually in the persuasion conditioning process. This operation is run on a different tier on Instagram and twitter

This embodiment comprises of the stated software and methodology according to claim 1Neuroalgorithmic Hypnosis, the Supporting studies have shown social media applications like Instagram can induce a cognitive trance-like hypnotic state within the neurological framework. This allows users to become more agreeable to ideas and advertising. When applied in neuroscience: psychiatry, cognitive operation of adopting ideas and understanding perspectives, thought processes and the alteration of one's baseline of comprehension within equilibrium and therefore, altering existence or actuality in induced state. The use of this methodology assists in digital psychological warfare propaganda which is prevalent and furthers the educational uses regarding neurological science.
Changing thought processes
Formulating directed perspective and understanding
Adopting innovative ideas and knowledge
Education and Treatment
Reconfirming baseline of understanding, equilibrium, existence and or actuality as a whole

“Their results showed the highest agreement between brain structure and brain function in areas forming part of the “default mode network”, which is associated with daydreaming, imagination, and self-referential thought. “In comparison to other networks, the default mode network uses the most direct anatomical connections. We think that neuronal activity is automatically directed to level off at this network whenever there are no external influences on the brain,” says Andreas Horn, lead author of the study and researcher in the Center for Adaptive Rationality at the Max Planck Institute for Human Development in Berlin.

Living up to its name, the default mode network becomes active in the absence of external influences. In other words, the anatomical structure of the brain has a built-in autopilot setting. It should not, however, be confused with an idle state. On the contrary, daydreaming, imagination, and self-referential thought are complex tasks for the brain.”

### Resource:

https://maxplanckneuroscience.org/brain-on-autopilot/

“Taken together, [studies show] internet addiction is associated with structural and functional changes in

brain regions involving emotional processing, executive attention, decision making, and cognitive control.”—research authors summarizing neuro-imaging findings in internet and gaming addiction

This embodiment comprises of the stated software and methodology according to claim 1, compromising the use behavioral isolation observation and specialized algorithm to determine computation results. Diagnostic Patch and Device is a wireless medical device and cylinder patch with a built-in hard drive for computing data calculations, wireless transmitter, and memory. This device contains a small built-in lab centrifuge and an automatic applicator with a built-in cartridge for two small needles: an injector and the sample collector. The Cylinder Diagnostic Patch process of monitoring blood levels and data continuously is a procedure intended for short term or prolonged use.

Cylinder Diagnostic Patch is attached to the arm, it is equipped with a tiny built-in lab expert that separates your samples into designated areas within the device, which first checks the quantity of red and white blood cells. Cylinder Diagnostic Patch has a built-in compact centrifuge machine to spin the blood so the blood cells can go down to the bottom of the tiny test tubes. This process calculates data regarding your metabolism, hormone levels and helps identify severe diseases such cancer. Cylinder Diagnostic Patch has an automatic applicator with a built-in cartridge for two small needles: an injector and the sample collector. The Myriad Cylinder Diagnostic Patch is placed on the patient's arm before the medical needles are prompted to dispatch. The applicator is equipped with a natural adhesive which holds the device firmly and comfortably in place. This device is water resistant.

The device has tiny sensors to alert the patient if the device becomes disconnected. In the event of the applicator becoming dismounted, the patient will have to remove the device completely, a new needle will have to be inserted after the injection site is cleaned. The patient can then reposition the device back into place. The Myriad Cylinder Diagnostic Patch device comes with additional medical needles. Your physician will receive the monitoring data and a copy will be sent to your phone. Data is sent by your transmitter. The transmitter sends real-time readings wirelessly to a receiver so you can view them on your smart phone. Diagnostic Patch Blood Sample Analysis. This process requires analyzing the blood sample components of modification sequences, levels, behavioral data during the process of patient use. Diagnostic Patch and Device's built-in Lab, prompts the process of comparative analysis processes, using the methodology of disease cataloging diagnostic detection computation as a prototype, the sequences attained from patient's samples can be quantified with the same metric as a baseline, thus producing laboratory diagnostic results. It is then grouped together into one blood panel with the results sent to a third-party laboratory for further comparative analysis of findings, then finally upon completion, then sent to physician with detailed report of patient findings.

### Diagnostic Analysis Process

Sample collections containing blood extracted from patients with vanous diseases are quantified and sequenced into categorical divisions which are computed into data analysis processes which can then identifiably classify diseases in cataloging diagnostic detection which is then is as a baseline in determining diagnostic results. Certain diseases and conditions can be detected by blood tests. Furthermore, they demonstrate how well your treatment works by checking the function of your organs. This device can be used for medicine distribution and disease management. Pharmaceutical Compounding Data collected from The Myriad Cylinder Diagnostic Patch will provide the opportunity to with customized medicine compounding. Comcode Distributor. The smart phone capabilities: updates, alarms alerts, and patient portal for results.ng the administrative access controls to the telecommunication layer content database, which can also operate manually in the persuasion conditioning process. This operation is run on a different tier on Instagram and twitter.

This embodiment comprises of the stated software and methodology according to claim 1, Homeland Security Sextortion Secure System is a software which verifies and monitors the integrity of user's electronic communication such as: alerts, notifications, and correspondence to counteract phishing attacks, malicious links and social engineered extortion vulnerabilities concerning children or persons with disability.

This filtration works in similar methodology as Gmail filtration system regarding spam. However, Homeland Security Sextortion Secure System ensures that outside emails, messages, content, or text messages never reaches the users server by immediately bouncing the transmission back to the original sender once its contents are classified as unsecure in the quarantine process or specified registered corresponded. The system monitors the algorithm to detect inappropriate content both outgoing and incoming. The system then rapidly dispatches its spider malware during the initiate “bounce” upon transmission, with the sole purpose of extracting data and user location from the sender, completely bypassing VPNs. This method is carried out by using the quarantine process within a third-party confinement, which is overseen by Homeland and National Security efforts. Upon transmission analysis, the data will be classified depending on the qualifications met within the specified categorical metric system ranging between 1-3 in severity, regarding unsecure communication or malicious content attached. The classification process consists of an in-depth A.I machine methodology, which detects phrasing, wording, monetary characterization, researched linguistic identification makers and extortion approaches, and location based automatic denial communication procedures in high-risks areas where phishing and social engineering extortion is prominent.

This classification process enables a rerouting system which redirects download attachments to a designated and segregated server to examine its contents. While simultaneously opening the attachment contents of the download, the system rapidly dispatches its spider malware upon the initial “bounce” transmission with the sole purpose of extracting the data and location of the sender for analysis to Homeland Security.

This embodiment comprises of the stated software and methodology according to claim 1, Disabling Bot Attacks and Infiltration of Propaganda Procedures for Department of Defense and Military Management. The disabling of bot attacks is essential in framework of military action in the scope of decreasing the distribution of harmful propaganda in the context of digital psychological warfare which is prevalent. Quarantining and immediate reactive shadow banning of accounts associated with rapid interactions, engagements, and posting with other accounts mirroring identical prerequisites. Disabling bot accounts rapid posting This embodiment comprises of the stated software and methodology according to claim 1, Integrated Sensory Constructure Unit American Financial Industrial Teller Security.

Major attacks attributed to the North Korean Lazarus group, where the primary goal of an attack was monetary, executed by installing malware which would then trigger a malfunction command in the software to distribute money the black box mechanism used in ATMs. These attacks would classify as logical and supply chain attacks, which were spearheaded by infecting the systems utility with malware.

Black box malware attacks where a second generation cyberattack to card skimming which had fallen significantly with the introduction of black box malware phishing. Reconstructing all physical teller machines with a base shell that is reinforced and intertwined with sensory cables within the infrastructure. The sensory cables would be an essential aspect of the overall constructure integrity of the machines, which would notify a security agent if tampered or altered in any way. The counter space and buttons would require built-in scanners which would activate a digital capture which could retrieve fingerprints and the constructure sensory system would notify the task force. Impersonation of technician with the purpose to withdraw can be prevented by encoding uniforms with primary identification codes and phantom codes, as mentioned in previous assignment that counter-syncs, records maintenance work and sends reports to security and manageable after the completion of each worksite.

The uniforms would be reimagined with unique coloring for added security and defense of the detection of imposters by unique colors, which activate a high-security responses. This helps protect the technicians as well.

Detection of digital devices within close range: logs, identified and classified security risk. In the case of laptops within close range, would signal an alarm to both user and security system. This would prompt the system to ask for additional security information to verify the identity. Software engineered for rapid and continuous loop system checks after, during, and before each transaction assuring the integrity of security system. This system would assist in the isolating attacks by early detection and swift retrieval of information regarding attacks. Reversing attack: Defense: upgrading sensory detection on USB ports and any vulnerable ports on machines which swipes the machines identifications to the security center and signals an alarm system. A locking mechanism arrest devices in place, making attackers unable to retrieve their devices.

This defense system would also install remote malware and phishing software to send the attackers data to law enforcement with the goal of compromising cybercriminals enterprises and their associates.

This embodiment comprises of the stated software and methodology according to claim 1, Phantom Banking Transaction System Data Protection Technology.

The updating financial industry system with Phantom Banking Transaction System, a data protection technology. The baseline execution in protecting an operating system with a crypto processor in place, as the financial industry is more vulnerable to being compromised, particularly, in the current trend of cryptocurrency. It requires current updates and/or patches to defend against such attacks.

The keystone of the security subsystem, eliminating the need to protect the rest of the subsystem with physical security measures and ensuring each system isolation within the framework of the finance industry. In addition to utilizing individually unique and auto-populated disposal firewalls, VPNs, and encryption infrastructure to ensure security. Assuring all anti-virus and configurations are current and up to date within the standard and practices. Implementing randomized auto-penetration testing throughout the workday to ensure the responsiveness and effectiveness of the security infrastructure in place within the department. Implementing a secured shuffling server system to complete each verified financial transaction to ensure further privacy during the confidential payment process by running this encryption simultaneously but perpendicularly as a single method for every transaction.

This system would create phantom transactions IDs with temporary codes that are to be assigned only at the completion of each transaction rather than standard and permanent routing numbers which can be compromised mid-transaction.

The daily reports of this process would be overseen, monitored, and assessed for quality security assurance by the security department. Lastly, isolating each susp1c10us behavior of foreign penetration attempts identified in real-time during this aggressive testing methodology.

Establishing that the financial industry has a higher risk of being compromised, it is recommended that you do not store your passwords on any of your devices, particularly mobile devices as they are often at higher risk and can be lost, human-caused error. Password complexity is essential for baseline user protection. It is not recommended to use birthdays, pet names or any information that can be easily retrieved by social media or from any of the various mass digital citizen directories online.

Research is key m both defense as well as for the attackers. Again, with readily user information available an attacker can obtain information by your content posted on your social media accounts or even google. Then moving on the second line of offense, social engineering, and phishing after that baseline of databases. User information can be bought and sold from dating sites and even retail, our digital footprints out there and the name of the game is data

This embodiment comprises of the stated software and methodology according to claim 1, function is digital caretaker for those specifically with Autism who struggle with executive functions but can be used for a wide variety of conditions/disables.

It is an interactive app which allows users to program their daily routines. The app is synchronized with a smart watch and device for the patient's home. The software can scan patients' faces and teeth for confirmation of function. It is a reminder for medication, exercise, executive functions, hygiene, and most importantly an interactive friendly companion. It uses facial recognition to assist with eye contact training.

The data distributor is sent to physicians, guardians, and caretakers. This system not only provides a comprehensive treatment for independent living but data that will be invaluable to a variety of diseases.

This embodiment comprises of the stated software and methodology according to claim 1, Wearable Medical Distributor. The distributor is an innovative medical device which computes compounds and codes. Much like a pacemaker, this device is installed on the body as hardware (i.e., Vagnus Nerve Stimulation implant). The device will be very compact in size, and its benefits include regulation and measurement of blood tests, and disease management for any number of medical conditions and chronic illnesses, including cancer, autoimmune disorders, and other diseases that require hands on medication management.

The device monitors the body to collect and formulate patterned data which is sent to a third-party location where the prescribing physician can view and manage remotely. It monitors and produces patterns of the body's cycle and distributes custom compounded medical dosages as PRN, exactly how you need it and when it needs it. Through this function, it can provide personalized management plans for patients based on height, weight, and other complex parameters.

Data will be collected and collated for tens of thousands of patients or more, organized by condition to provide useful information to feed algorithmic calculations that ensure the continuous improvement of the underlying software. The data will be private, and protected by HIPPA laws, and it will be decoded to allow contributions to innovative textbooks and learning tools for students in the medical field. The information can also be utilized for case studies and study guides in classroom settings. The constant flow of data would be continually updated and refreshed, and the information would be kept in a shared database or digital environment for use across different platforms.

This method will revolutionize the medicinal and pharmaceutical industry, as it can decrease the amount of drug abuse and accidental overdosing. It allows physicians to have a 365 scope of the patient's makeup and diseases. With the data collected by physicians can monitor more closely how a disease progresses over time for a single patient or a collective. Most importantly, this can be used as a diagnostic tool, for further education, and preventive medicine, by capturing images and data in ways which customized and revolutionized medication.

In summary, the distributor is a new form of communication in the medical industry. It can be utilized as an effective diagnostics tool that uses a dynamic digital medical dictionary. The integration of Wi-Fi and Bluetooth technology will allow communication between consulting physicians and medical professionals to assist with complex treatment plans.

This embodiment comprises of the stated software and methodology according to claim 1, secure Biometrics Boarding procedures requiring passengers to complete a biometric scan upon purchasing travel tickets, different from an ID or Passport. This procedure enforcing a digitally encrypted security screen, inscribed with unique and phantom coding specific to the enterprise thus making the biometrics information requiring a specific matching key for decryption, analysis, and confirmation, thus, in the case of hacking, this information cannot be compromised. The collection of biometrics data and the security controls in place to protect this data are of grave concern to groups like the Electronic Privacy Information Center (EPIC). Secure Biometrics Boarding procedure mandatory scans to board any aircraft or transportation network in the United States. This would ensure that every person is not only accounted for, but also meets the required credentials to board any aircraft or otherwise. These requirements would be extended to the pilots, flight attendants and all airport personnel.

This methodology will be required in various stages during both domestic and international traveling, checking in, boarding, dismounting the aircraft and baggage claim. It is critical for Domestic flights to adhere by these enforcements to identify individuals who have entered the country illegally with the intention of committing terrorism or other crimes.

This technology and procedure are to be used alongside the Biometric Facial Comparison Technology already in place with the US Customs and Border Protection.

This technology could prevent attacks such as high-jackings, fugitives, mlssmg children, criminal enterprises, or unregistered persons boarding any means of public transportation (Air, Land and Sea environments).

Biometrics logging facial structure associated with high risks regions or enemy territory, assisting with the everyday data scans already in place through routine biometrics surveillance.\

This embodiment comprises of the stated software and methodology according to claim 1, Federal Safe Mobile Carrier Compliance Preventing Child Exploitation. Federal policy and practice standards to be updated with compliance of all mobile carriers requiring that users complete cyber-safety training and user safe compliance agreement upon registration of all devices. User confidentiality is of the utmost importance, which is why. A.I. smart machine is the first line of defense in protecting citizen privacy in the fight against child exploitation.

### Processes and Procedures

All mods must have corresponding agreement and permission with digital signatures with/from legal guardians, who are also required to read an overview and answer questions regarding carrier and device agreements with software in effort of preventing child exploitation.

Users who are minors are required to have an added encryption and filter feature which prevents nudes and/or private credentials from being distributed or received by an additional encrypted interception which auto flags high-risk content with utilizing smart A.I machine software download, required upon registration in all minor operated devices. Telecommunication efforts comply by monitoring numbers who engage m abnormal behavioral activities at rapid pace, packet flooding, denial of services, spamming, catfishing, and other cyber criminalities.

Detecting mobile devices who exchange large quantity of high-risk content concerning nudity, particularly in minor registered devices and credentials.

Those numbers are to be flagged for monitoring and detained in federal quarantine for further inspection.

Numbers registered with suspicious area codes and pinging from towers outside of approved geographical area of the minor's location are automatically flagged, in an effort in preventing sextortion, kidnapping, cyberbullying, and sex-trafficking.

These suspicious numbers are held in federal quarantine where reserve engineering can be engaged, by distributing Spider Malware for additional monitoring and inspections.

This process and procedures aid in establishing the sourcing and identifying the players within these criminal enterprises in the child trafficking, extortion, and overall exploitation.

Cellphone carriers should also auto-populate suicide resources anytime the word is mentioned in text exchange.

Critical operational tactic and precision of execution is non-negotiable. This requires forethought and planning but there's little room for trial and error in these ever-changing parameters. Response is everything in cyber-security, it is the defense and offense in the realm of real life, which is no game.

This embodiment comprises of the stated software and methodology according to claim 1, Biometric Scan for Firearm Purchase and Delivery. Biometrics Scan for Firearm Purchase and Delivery Compliance Act Procedures requiring customers to complete a biometric scan upon purchasing firearms, in addition to presenting an ID or Passport, to complete transaction, both when purchasing and upon delivery with a verified digital biometric signature. This procedure enforcing a digitally encrypted security screen, inscribed with unique and phantom coding specific to the enterprise thus making the biometrics information requiring a specific matching key for decryption, analysis, and confirmation, thus, in the case of hacking, this information cannot be compromised. The collection of biometrics data and the security controls in place to protect this data are of grave concern to groups like the Electronic Privacy Information Center (EPIC). Biometrics Scan upon Delivery is mandatory to complete all firearms transactions in the United States. This would ensure that every person is not only accounted for, but also meets the age requirement, confirming the identity of card holder and other credentials required to purchase firearms.

All vendors must comply to be an authorized distributor of firearms under federal regulation and standard practices. This technology and procedure are to be used alongside the Biometric Facial Comparison Technology already in place with the US Customs and Border Protection both purchasing and delivery. This technology could prevent the buying of both legal and illegal firearms by children and other authorized purchasing with stolen credit cards.

This embodiment comprises of the stated software and methodology according to claim 1, Cryptographic Streaming. Cryptographic Streaming is the second generation of encryption and decryption methodology used in teleconferencing. Instead of utilizing a primary shared encryption key, this method requires software which is engineered for rapid and continuous encryption looping within a system for the purpose of cryptographic checks during government telecast, financial transactions, and antiterrorism protocol, assuring the integrity of security system enterprise.

Cryptographic Streaming would assist in isolating attacks, intrusions, fraud, terrorism detection and eavesdropping by early detection and swift retrieval of information regarding attacks as seen in the Integrated Sensory Constructure Unit, within the financial industry. This procedure would also incorporate the enforcement of digital encryption security screen, inscribed with unique and phantom coding specific to enterprise, thus, making the biometric information requiring a specific matching key for decryption in the same steaming methodology, analysis, and confirmation, thus, in the case of hacking, this information cannot be compromised nor accessed.

It would be required of employees working in high-risk industries, such as The Department of Homeland Security and The Department of Defense to register biometric data to participate future telecast etc., as an essential part of policy and procedures within enterprise. The collection of biometric data and the security controls in place would be to protect data and privacy, most notably suggested with, Secure Biometrics Boarding at Boarder Control. The disadvantage with using symmetric encryption is always interception. The use of Cryptographic streaming and shuffling encryption makes it impossible to hack a code which is rapidly changing the series of temporary encryptions without pattern or sequence.

This methodology would also be utilized in Next Generation Cryptographic regarding text messaging and e-mail, it is a critical baseline in protecting the confidentiality, integrity, and availability in the framework of the overall Nation Security. Cryptographic Streaming constantly resetting the encryption output ensures that there are no sequences and codes, which historically were essential but now arbitrarily obsolete to intrusion.

This embodiment comprises of the stated software and methodology according to claim 1, Self-Assisted Driving Mode with Frequency. The assurance automobile safety is dependent up the Frequency Communication system as it can give an estimation of frequency traffic, meaning not only can sensors put up the wireless frequency but also the frequency installed within stop lights and cell phone devices to prevent accidents, both vehicular and human casualty. This system offers precision estimation data within the mapping and GPS system due to the AnonemiX frequencies. Media Frequency is the concept of utilizing the power of a dual combined radio frequency vacuum continuum which transmits signals pinging from the nearest cell phone user's antenna to transmit necessary elements to trigger looping of frequencies to power a communication network with built-in fibers within cellular devices.

Devices equipped with dual electromagnetic radio wave acting as an input and output transmission to be routed to ping from users as well as efficiency portable cell towers. This mechanism is an exterior feature which has no access to the internal network which would be transmitted on a separate frequency. An efficiency portable cell tower adaptor which can be powered by existing power sources, such as preexisting power beams, which hardware would then be installed on to poles. This combined radio frequency vacuum is to be used with the intention of powering personal. Communication such as email, text, video, audio communication and general internet access.

This concept works in the event of natural disasters, providing internet access in low-income areas, in addition to portable cell towers which can be made from recyclable and biodegradable materials. The more people using the cell service carriers the stronger the frequency, which creates an incentive for both customers and carriers. Carriers and manufacturers can also provide low-income areas with portable routers to strengthen the network frequency. Again, this can be installed upon existing power poled equipped an efficiency portable cell tower adaptor, offering internet access in rural areas. This cost-effective methodology can be used throughout the USA, and adopted world-wide which provides additional source opportunity for speed, as well as the adaptors providing a sleeker addon aesthetic.

This embodiment comprises of the stated software and methodology according to claim 1, Media Frequency. The concept of utilizing the power of a dual combined radio frequency vacuum continuum which transmits signals pinging from the nearest cell phone user's antenna to transmit necessary elements to trigger looping of frequencies to power a communication network with built-in fibers within cellular devices.

Devices equipped with dual electromagnetic radio wave acting as an input and output transmission to be routed to ping from users as well as efficiency portable cell towers. This mechanism is an exterior feature which has no access to the internal network which would be transmitted on a separate frequency.

An efficiency portable cell tower adaptor which can be powered by existing power sources, such as pre-existing power beams, which hardware would then be installed on to poles.

This combined radio frequency vacuum is to be used with the intention of powering personal communication such as email, text, video, audio communication and general internet access.

This concept works in the event of natural disasters, providing internet access in low-income areas, in addition to portable cell towers which can be made from recyclable and biodegradable materials. The more people using the cell service carriers the stronger the frequency, which creates an incentive for both customers and carriers.

Carriers and manufacturers can also provide low-income areas with portable routers to strengthen the network frequency. Again, this can be installed upon existing power pole equipped with an efficient portable cell tower adaptor, offering internet access in rural areas.

This cost-effective methodology can be used throughout the USA, and adopted world-wide which provides additional source opportunity for speed, as well as the adaptors providing a sleeker add-on aesthetic.

This embodiment comprises of the stated software and methodology according to claim 1, Bank-Comm Secure Systems is a software which verifies and monitors the integrity of user's electronic communication such as: alerts, notifications, and correspondence to counteract phishing attacks, malicious links, and social engineered extortion vulnerabilities concerning financial institutions or persons. This filtration works in similar methodology as Gmail filtration system regarding spam. However, Bank-Comm Secure Systems ensures that the email never reaches the users server by immediately bouncing the transmission back to the original sender once its contents is classified as unsecure in the quarantine process. The system then rapidly dispatches its spider malware during the initiate “bounce” upon transmission, with the sole purpose of extracting data and user location from the sender, completely bypassing VPNs. This method is carried out by using the quarantine process within a third-party confinement, which is overseen by Homeland and National Security efforts. Upon transmission analysis, the data will be classified depending on the qualifications met within the specified categorical metric system ranging between 1-3 in severity, regarding unsecure communication or malicious content attached. The classification process consists of an in-depth A.I machine methodology, which detects phrasing, wording, monetary characterization, researched linguistic identification makers and extortion approaches, and location based automatic denial communication procedures in high-risks areas where phishing and social engineering extortion is prominent. This classification process enables a rerouting system which redirects download attachments to a designated and segregated server to examine its contents. While simultaneously opening the attachment contents of the download, the system rapidly dispatches its spider malware upon the initial “bounce” transmission with the sole purpose of extracting the data and location of the sender for analysis to Homeland Security.

This embodiment comprises of the stated software and methodology according to claim 1, Detection Software uses A.I machining and digital recognition to analyze linguistics data and patterning for classification criterion for detecting propagandist and terrorism paraphernalia in all language phrasing but also graphical delivery.

System Detection Software is engineered for administrator access use, encompassing most social media platforms and applications as a mandated government compliance initiative towards fighting terrorism, specifically, psychological cyber warfare.

In prohibiting unnecessary misclassification and quarantine holdings, the software operates with IAM/PAM access control methodology procedural tactics. Enforcing security monitoring and procedural processes by segregated location mapping, which concentrates on high-risk territories and associated enterprises, utilizing the cyber security surveillance systems of Cyber-Micro-Expressions Computation quarantine server methodology.

This embodiment comprises of the stated software and methodology according to claim 1, Interceptive Communication Integration Morse Codex Systems is formulated to intercept social media telecommunications messaging through platform stories.

The account stories can then be intercepted and used as vehicles to rely on two-way communication sequencing.

The process of using a similar format to the current methodology now in place, located in the account's privacy settings for delegation of authorization views. However, in the case of Interceptive Communication Integration (Morse Codex Systems) the output distributed is operated on an entirely different server. This requires a randomized sequencing output transmission which assists in avoiding detection.

The algorithm then organizes intelligences information in the proper order, per sequence deliverance upon transmitted intel, from sender to recipient.

This embodiment comprises of the stated software and methodology according to claim 1, Interactive Integration of Artificial Intelligence is an advanced learning methodology in which an operating system alternatively and cognitively trains the learner to think in the same sequences in which the A.I instructions is computing for cognizance.

This procedural processing is designed to run in addition to the A.I processes of recording the subject's inputting data theorem to perform in a synchronous awareness. Thus, achieving full scale cognitive configuration recollection for human development and processes. Monitoring the frequency level capacity to detect abnormalities as seen in hackings and network system server monitoring to collect intelligence. Particularly in IOTs. For example, with physical access to Peloton bikes, one could access these devices, including their camera and microphone. Malicious apps could be added to Peloton disguised as Netflix or Spotify, and victims could be encouraged to enter their credentials through those apps. Thus, monitoring the frequencies can detect if additional bandwidth is used in the overall measurement within the capacity which has been allotted, thus this would be an indication of hacking and monitoring.

This embodiment comprises of the stated software and methodology according to claim 1, Diagnostic Patch and Device is a wireless medical device and cylinder patch with a built-in hard drive for computing data calculations, wireless transmitter, and memory. This device contains a small built-in lab centrifuge and an automatic applicator with a built-in cartridge for two small needles: an injector and the sample collector. The Cylinder Diagnostic Patch process of monitoring blood levels and data continuously is a procedure intended for short term or prolonged use.

Cylinder Diagnostic Patch is attached to the arm, it is equipped with a tiny built-in lab expert that separates your samples into designated areas within the device, which first checks the quantity of red and white blood cells. Cylinder Diagnostic Patch has a built-in compact centrifuge machine to spin the blood so the blood cells can go down to the bottom of the tiny test tubes. This process calculates data regarding your metabolism, hormone levels and helps identify severe diseases such cancer. Cylinder Diagnostic Patch has an automatic applicator with a built-in cartridge for two small needles: an injector and the sample collector. The Myriad Cylinder Diagnostic Patch is placed on the patient's arm before the medical needles are prompted to dispatch. The applicator is equipped with a natural adhesive which holds the device firmly and comfortably in place. This device is water resistant.

The device has tiny sensors to alert the patient if the device becomes disconnected. In the event of the applicator becoming dismounted, the patient will have to remove the device completely, a new needle will have to be inserted after the injection site is cleaned. The patient can then reposition the device back into place. The Myriad Cylinder Diagnostic Patch device comes with additional medical needles. Your physician will receive the monitoring data and a copy will be sent to your phone. Data is sent by your transmitter. The transmitter sends real-time readings wirelessly to a receiver so you can view them on your smart phone. Diagnostic Patch Blood Sample Analysis. This process requires analyzing the blood sample components of modification sequences, levels, behavioral data during the process of patient use. Diagnostic Patch and Device's built-in Lab, prompts the process of comparative analysis processes, using the methodology of disease cataloging diagnostic detection computation as a prototype, the sequences attained from patient's samples can be quantified with the same metric as a baseline, thus producing laboratory diagnostic results. It is then grouped together into one blood panel with the results sent to a third-party laboratory for further comparative analysis of findings, then finally upon completion, then sent to physician with detailed report of patient findings.

### Diagnostic Analysis Process

Sample collections containing blood extracted from patients with vanous diseases are quantified and sequenced into categorical divisions which are computed into data analysis processes which can then identifiably classify diseases in cataloging diagnostic detection which is then is as a baseline in determining diagnostic results. Certain diseases and conditions can be detected by blood tests. Furthermore, they demonstrate how well your treatment works by checking the function of your organs. This device can be used for medicine distribution and disease management. Pharmaceutical Compounding Data collected from The Myriad Cylinder Diagnostic Patch will provide the opportunity to with customized medicine compounding. Comcode Distributor. The smart phone capabilities: updates, alarms alerts, and patient portal for results.

This embodiment comprises of the stated software and methodology according to claim 1, The A.I Doppler Human Ape Interface Data System (AdHaIDs) monitors the capacity frequencies to detect atmospheric conditions m space for potential adaptation, research, exploration, and habitation. This system is several units meant to be deployed in various locations for prior mention purposes regarding missions. The Free xUnit A.I Doppler Human Ape Interface Data System, separate from the stationary deployed A.I Doppler Human Ape Interface Data System aviates openly, collecting data while using frequencies to guild the doppler into territories which mimic required conditions for habitation and desired research data.

Once the Free xUnit A.I Doppler Human Ape Interface Data Systems has detected location of interest (AdHaIDs) is then dispatched for stationary based research data collection for the proximation of a year to record adaptation, research, exploration, and habitation. The A.I Doppler Human Ape Interface Data System is equipped with adaptation collections capabilities, camera's, mimics human anatomy with the use of A.I to determine habitation possibility and resource, sound recording, protective base, and Camascibe for reporting data into file formatting for finale review to space station for researchers.

This embodiment comprises of the stated software and methodology according to claim 1, compromising the use behavioral isolation observation and specialized algorithm to determine computation results. Every branch of law enforcement is required to use IAForce Software. This software has a shared database that is powered by A.I., which still mandates officer oversight. Access controls for differ between agencies. This database allows communication and cross-referencing work to run smoother within agencies, allowing access to detailed information regarding crimes, evidence, and suspects. IAForce offers Retroactive A.I. Evidence linking of prior digital evidence which required advancements which were not available at the time, such as in cases with DNA evidence.

This embodiment comprises of the stated software and methodology according to claim 1, compromising the use behavioral isolation observation and specialized algorithm to determine computation results. Wearable Medical Distributor.

The distributor is an innovative medical device which computes compounds and codes. Much like a pacemaker, this device is installed on the body as hardware (i.e., Vagnus Nerve Stimulation implant). The device will be very compact in size, and its benefits include regulation and measurement of blood tests, and disease management for any number of medical conditions and chronic illnesses, including cancer, autoimmune disorders, and other diseases that require hands on medication management.

The device monitors the body to collect and formulate patterned data which is sent to a third-party location where the prescribing physician can view and manage remotely. It monitors and produces patterns of the body's cycle and distributes custom compounded medical dosages as PRN, exactly how you need it and when it needs it. Through this function, it can provide personalized management plans for patients based on height, weight, and other complex parameters.

Data will be collected and collated for tens of thousands of patients or more, organized by condition to provide useful information to feed algorithmic calculations that ensure the continuous improvement of the underlying software. The data will be private, and protected by HIPPA laws, and it will be decoded to allow contributions to innovative textbooks and learning tools for students in the medical field. The information can also be utilized for case studies and study guides in classroom settings. The constant flow of data would be continually updated and refreshed, and the information would be kept in a shared database or digital environment for use across different platforms.

This method will revolutionize the medicinal and pharmaceutical industry, as it can decrease the amount of drug abuse and accidental overdosing. It allows physicians to have a 365 scope of the patient's makeup and diseases. With the data collected by physicians can monitor more closely how a disease progresses over time for a single patient or a collective. Most importantly, this can be used as a diagnostic tool, for further education, and preventive medicine, by capturing images and data in ways which customized and revolutionized medication.

In summary, the distributor is a new form of communication in the medical industry. It can be utilized as an effective diagnostics tool that uses a dynamic digital medical dictionary. The integration of Wi-Fi and Bluetooth technology will allow communication between consulting physicians and medical professionals to assist with complex treatment plans.

Although the teaching of the present application has been described in detail for the purpose of illustration, it is understood.

An embodiment of compromising software with a critical interface consists of an algorithm that records micropatterns of isolated human behavior, content, and critical elements of tactical process procedures within various sections of the present application, which require cyber-microexpression for computation. Quantum Sequence Triplication is the use to achieve Quantum Supremacy without grey area bias.

1. A software process and methodology according to claim 1, wherein compromising of a critical interface consisting of a system which records micropatterns of isolated human behavior, content, and tactical process procedures regarding various sectors with a specialized algorithmic database and Cyber-Micro-Expressions. resulting in The Alma Project, a neurological trauma treatment for treating complex post-traumatic stress syndrome in patients with Chronic Medical, Mental Health conditions and with Veterans Trauma Recovery. This program is for treating veterans, refugees, and victims of violent, war or sex crimes.
